(19) United States
(12) Patent Application Publication
(10) Pub. No.: US 2023/0416687 A1
(43) Pub. Date: Dec. 28, 2023

(54) LUNG CANCER MODELING USING NORMAL LUNG ORGANOID WITH LUNG CANCER CELLS

(71) Applicant: THE CATHOLIC UNIVERSITY OF KOREA INDUSTRY-ACADEMIC COOPERATION FOUNDATION, Seoul (KR)

(72) Inventors: Sung Won KIM, Seoul (KR); Ayoung JO, Seoul (KR); Min Jae LIM, Gyeonggi-do (KR); Jung Eun LEE, Gyeonggi-do (KR); Seung Ho YANG, Seoul (KR)

(21) Appl. No.: 18/342,930

(22) Filed: Jun. 28, 2023

(30) Foreign Application Priority Data
Jun. 28, 2022 (KR) 10-2022-0078899
May 30, 2023 (KR) 10-2023-0069304

(51) Int. Cl. C12N 5/071

(52) U.S. Cl. CPC C12N 5/0688; C12N 2502/30; C12N 2503/02

(57) ABSTRACT

The present invention relates to a lung cancer model based on a normal lung organoid, prepared by fusing cancer cells to the normal lung organoid. By using the lung cancer model according to the present invention, it is possible not only to observe the progression or metastasis pattern of lung cancer from the early stage of proliferating lung cancer cells to the stage of forming a tumor but also to compare the effect of an anticancer agent at each stage, and thus the information on an appropriate therapeutic agent may be provided according to the stage of cancer development.

In addition, since the lung cancer model according to the present invention includes a normal lung organoid and cancer cells, which are fused, it can be effectively used to confirm the effect of an anticancer agent on normal tissue and the effect of cancer cell-specific apoptosis. In addition, since a conventional method of injecting human lung cancer cells into a mouse has a problem in that unpredictable reactions are caused by immune responses or the like, the present invention is expected to be effectively used as a human lung cancer model.

### STATEMENT REGARDING GOVERNMENT RIGHTS

The present invention was undertaken with the support of: 1) Development of standardized organoid production technology based on airway stem cells and airway specific induced pluripotent stem cells with inkjet 3D bioprinting, National Research Foundation of Korea (NRF) grant No. 2019M3A9H2032424 funded by the Korea government (MSIT); 2) Study of mechanism and characterization of cultured alveolar organoid from lung dissociated cells in lung dECM, National Research Foundation of Korea (NRF) grant No. 2021R1I1A1A01059764 funded by the Ministry of Education; 3) Identification of glioblastoma-friendly microenvironment using patient-derived brain organoids and development of novel models using coculture of brain organoids and glioma stem cells, National Research Foundation of Korea (NRF) grant No. 2022R1A2C1007556 funded by the Korea government (MSIT); and 4) Development of therapeutic targets for brain metastasis via identification of genetic alterations associated with brain metastasis and modulation of brain microenvironment, National Research Foundation of Korea (NRF) grant No. 2021R1C1C2010469 funded by the Korea government (MSIT).

### CROSS-REFERENCE TO RELATED APPLICATION

This application claims priority to and the benefit of Korean Patent Application No. 10-2022-0078899, filed on Jun. 28, 2022, and Korean Patent Application No. 10-2023-0069304, filed on May 30, 2023, the disclosure of which is incorporated herein by reference in its entirety.

### BACKGROUND

### 1. Field of the Invention

The present invention relates to an organoid system that can provide a lung cancer model based on a lung organoid, prepared by fusing lung cancer cells to a normal lung organoid, and a use thereof.

### 2. Discussion of Related Art

Currently, animal models are widely used for disease models, and since animals are different from humans in terms of physiology, there are limitations in realizing human diseases in animals, and ethical issues are steadily emerging.

Organoids are three-dimensional cell constructs, and are recently attracting attention in that they consist of various cells constituting tissue, and thus can mimic in vivo environments. Accordingly, organoids are being actively studied and utilized in various areas ranging from basic biology research fields to various applied research fields such as novel drug development, disease modeling, and regenerative therapy.

Particularly, organoid models are in the limelight because they enable mechanistic research on diseases and patient-specific diagnosis for diseases using an organoid model manufactured of patient-derived cells.

Meanwhile, lung diseases caused by genetic factors, excessive smoking, fine dust generated by air pollution, viruses, and bacteria are one of the leading causes of death globally. However, due to the absence of a disease model, it is not easy to study the mechanism of lung diseases, and is also difficult to develop treatment methods therefor. Accordingly, there is an urgent need for the development of an in vitro model of lung disease, which can precisely mimic a human lung disease.

### RELATED ART DOCUMENT

### Patent Document

(Patent Document 0001) Korean Patent No. 10-2152492

### SUMMARY OF THE INVENTION

As a result of intensive research to develop an in vitro lung cancer model that can confirm the progression and metastasis of lung cancer, and an effect of an anticancer agent, the present inventors constructed a lung cancer model based on a normal lung organoid by fusing various cancer cells, including lung cancer cells, to the normal lung organoid, and thus completed the present invention.

Therefore, the present invention is directed to providing a method of preparing a lung cancer model using a normal lung organoid, which includes co-culturing a normal lung organoid and cancer cells.

The present invention is also directed to providing a lung cancer model prepared by the method of preparing a lung cancer model.

The present invention is also directed to providing a method of evaluating the efficacy of a lung cancer therapeutic agent.

The present invention is also directed to providing a method of screening a candidate material for a lung cancer therapeutic agent.

However, technical problems to be solved in the present invention are not limited to the above-described problems, and other problems which are not described herein will be fully understood by those of ordinary skill in the art from the following descriptions.

To achieve the purpose of the present invention, the present invention provides a method of preparing a lung cancer model using a normal lung organoid, which includes co-culturing a normal lung organoid and cancer cells.

In one embodiment of the present invention, the cancer may be one or more selected from the group consisting of breast cancer, colorectal cancer, lung cancer, small cell lung cancer, stomach cancer, liver cancer, blood cancer, bone cancer, pancreatic cancer, skin cancer, head and neck cancer, skin melanoma, intraocular melanoma, uterine sarcoma, ovarian cancer, rectal cancer, anal cancer, fallopian tube cancer, endometrial cancer, cervical cancer, vulvar cancer, vaginal cancer, esophageal cancer, small intestine cancer, endocrine cancer, thyroid cancer, parathyroid cancer, adrenal cancer, soft tissue tumors, female urethral cancer, penile cancer, prostate cancer, bronchial cancer, nasopharyngeal cancer, larynx cancer, chronic or acute leukemia, lymphocytic lymphoma, bladder cancer, kidney cancer, ureter cancer, renal cell cancer, renal pelvic cancer, central nervous system (CNS) tumors, primary CNS lymphoma, myeloma, brainstem gliomas, and pituitary adenomas, but the present invention is not limited thereto.

In another embodiment of the present invention, the cancer cells may be human primary cancer cells or established cancer cells, but the present invention is not limited thereto.

In still another embodiment of the present invention, the established cancer cells may be one or more selected from the group consisting of HeLa cells, COLO-201 cells, TE-1 cells, TE-2 cells, TE-3 cells, TE-7 cells, TE-8 cells, TE-12 cells, TE-13 cells, NRC-12 cells, MKN-45 cells, AGS cells, KATO-III cells, Hs746t cells, NCI-N87 cells, HEp-2 cells, Detroit 562 cells, HEC-1B cells, Hs-578T cells, K-562 cells, SF-188 cells, SNU-1 cells, H1299 cells, and WiDr cells, but the present invention is not limited thereto.

In yet another embodiment of the present invention, the lung organoid and the cancer cells may be mixed in a ratio of 1:1 to 1,000 cells, but the present invention is not limited thereto.

In yet another embodiment of the present invention, the preparation method may further include mixing the normal lung organoid and the cancer cells before co-culture and centrifuging the mixture, but the present invention is not limited thereto.

In addition, the present invention provides a lung cancer model prepared by the method of preparing a lung cancer model.

In one embodiment of the present invention, in the lung cancer model, lung cancer may be primary lung cancer or metastatic lung cancer, but the present invention is not limited thereto.

In addition, the present invention provides a method of evaluating the efficacy of a lung cancer therapeutic agent, which includes the following steps:
(a) treating a lung cancer model according to the present invention with a lung cancer therapeutic agent; and
(b) when the growth inhibition or apoptosis of the lung cancer model of (a) is increased compared to a control model, determining that the efficacy of the lung cancer therapeutic agent is excellent.

In addition, the present invention provides a method of screening a candidate material for a lung cancer therapeutic agent, which includes the following steps:
(a) treating the lung cancer model according to the present invention with a candidate material for a lung cancer therapeutic agent; and
(b) when the growth inhibition or apoptosis of the lung cancer model of (a) is increased compared to a control model, determining the candidate material as a lung cancer therapeutic agent.

In one embodiment of the present invention, the growth inhibition or apoptosis of the lung cancer model may be confirmed through the increase/decrease in area of the lung cancer model, but the present invention is not limited thereto.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIGS. 1 to 3 are images of lung cancer models prepared by fusing lung cancer cells to normal lung organoids derived from different people; and

FIG. 4 is a set of images illustrating the results that confirm the expression of alveolar-specific markers, HT1-56, HT2-280 and ColA1, in lung cancer models prepared by fusing lung cancer cells to normal lung organoids.

### DETAILED DESCRIPTION OF EXEMPLARY EMBODIMENTS

Each description and embodiments disclosed herein may be applied to other descriptions and embodiments. That is, all combinations of various elements disclosed herein are included in the scope of the present invention. In addition, it should not be seen that the scope of the present invention is limited by the specific description described below.

In addition, terms not specifically defined herein will be understood to have meanings conventionally used in the art to which the present invention belongs. In addition, unless particularly defined otherwise in the context, the singular includes the plural, and the plural includes the singular.

As a result of intensive research to develop an in vitro lung cancer model that can confirm the progression and metastasis of lung cancer, and an effect of an anticancer agent, the present inventors constructed a lung cancer model based on a normal lung organoid by fusing various cancer cells, including lung cancer cells, to the normal lung organoid, and thus completed the present invention.

Hereinafter, the present invention will be described in detail.

The present invention provides a method of preparing a lung cancer model using a normal lung organoid, which includes co-culturing a normal lung organoid and cancer cells.

In addition, the present invention provides a lung cancer model prepared by the method of preparing a lung cancer model.

The “lung cancer model” used herein is prepared by fusing cancer cells to a normal lung organoid, and refers to a lung cancer model that can confirm a pattern of progression of cancer from the beginning or a pattern of metastasis of cancer other than lung cancer to the lungs according to the type of bound cancer cell.

The “organoid” used herein is a cell aggregate made by aggregating/recombining cells isolated from stem cells or organ-derived cells through 3D culture, and may include an organoid or cell cluster formed from suspension cell culture. The organoid may also be referred to as a tiny organ mimic, an organ analogue, or a similar organ. The organoid may specifically include one or more types of cells among various types of cells constituting an organ or tissue, and should be able to reproduce the shape and function of the tissue or organ. According to one embodiment of the present invention, the lung organoid may be prepared using type 2 alveolar cells isolated from lung tissue, but the present invention is not limited thereto. The lung tissue may be derived from a human who does not have cancer.

The “normal” used herein refers to a non-malignant tumor or a negative state for malignancy, and includes a perfectly normal state without a disease, and a state corresponding to a diseased state other than a malignant tumor (cancer).

In the present invention, the cancer may be one or more selected from the group consisting of breast cancer, colorectal cancer, lung cancer, small cell lung cancer, stomach cancer, liver cancer, blood cancer, bone cancer, pancreatic cancer, skin cancer, head and neck cancer, skin melanoma, intraocular melanoma, uterine sarcoma, ovarian cancer, rectal cancer, anal cancer, fallopian tube cancer, endometrial cancer, cervical cancer, vulvar cancer, vaginal cancer, esophageal cancer, small intestine cancer, endocrine cancer, thyroid cancer, parathyroid cancer, adrenal cancer, soft tissue tumors, female urethral cancer, penile cancer, prostate cancer, bronchial cancer, nasopharyngeal cancer, larynx cancer, chronic or acute leukemia, lymphocytic lymphoma, bladder cancer, kidney cancer, ureter cancer, renal cell cancer, renal pelvic cancer, central nervous system (CNS) tumors, primary CNS lymphoma, myeloma, brainstem gliomas, and pituitary adenomas, but the present invention is not limited thereto.

The “cancer tissue” and “cancer cells” used in the specification are used as the same concepts as “tumor tissue” and “tumor cells.”

In the present invention, the cancer cells may be primary cancer cells or established cancer cells collected from an early cancer patient, but the present invention is not limited thereto.

The “primary cancer cells” used herein refer to cancer cells collected from a patient having stage 1 or 2 cancer, which is an early stage of malignant tumors or cancer.

The “established cancer cell” used herein refers to a cancer cell line sold in the art, and may include any cancer cell line sold in the art without limitation.

In the present invention, the established cancer cells may be one or more selected from the group consisting of HeLa cells, COLO-201 cells, TE-1 cells, TE-2 cells, TE-3 cells, TE-7 cells, TE-8 cells, TE-12 cells, TE-13 cells, NRC-12 cells, MKN-45 cells, AGS cells, KATO-III cells, Hs746t cells, NCI-N87 cells, HEp-2 cells, Detroit 562 cells, HEC-1B cells, Hs-578T cells, K-562 cells, SF-188 cells, SNU-1 cells, H1299 cells, and WiDr cells, but the present invention is not limited thereto.

Among the established cancer cells, HeLa cells are derived from human cervical squamous cell carcinoma, COLO-201 cells are derived from human colorectal cancer, TE-1, 2, 3, 7, 8, 12 and 13 are derived from human esophageal cancer, NRC-12 cells are derived from human renal cell carcinoma, MKN-45 cells, AGS cells, KATO-III cells, Hs746t cells and NCI-N87 cells are derived from human gastric cancer, HEp-2 cells are derived from squamous cell carcinoma of the human larynx, Detroit 562 cells are derived from human pharynx cancer, HEC-1B cells are derived from human endometrial cancer, Hs-578T cells are derived from human breast cancer, K-562 cells are derived from human chronic myelogenous leukemia, SF-188 cells are derived from human brain cancer, SNU-1 cells are derived from human gastric cancer, H1299 cells are derived from human lung cancer, or WiDr cells are derived from human colorectal cancer.

In the present invention, the lung organoid and the cancer cells may be mixed in a ratio of 1:1 to 1000 cells, 1:1 to 800 cells, 1:1 to 500 cells, 1:1 to 300 cells, 1:1 to 100 cells, 1:1 to 80 cells, 1:1 to 50 cells, 1:1 to 30 cells, 1:1 to 10 cells, or 1:1 cells, but the present invention is not limited thereto. The lung organoid and the cancer cells may be mixed in an appropriate ratio by one of ordinary skill in the art depending on a desired lung cancer model. According to one embodiment of the present invention, the lung organoid and the cancer cells may be mixed in a ratio of 1:100 cells.

In the present invention, the lung organoid and the cancer cells may be mixed in a ratio of 1:1 to 1000, 1:1 to 800, 1:1 to 500, 1:1 to 300, 1:1 to 100, 1:1 to 80, 1:1 to 50, 1:1 to 30, 1:1 to 10, or 1:1, but the present invention is not limited thereto. The lung organoid and the cancer cells may be mixed in an appropriate ratio by one of ordinary skill in the art depending on a desired lung cancer model. According to one embodiment of the present invention, the lung organoid and the cancer cells may be mixed in a ratio of 1:100.

In addition, a culture medium used in co-culture in the present invention is not particularly limited, but culturing may be performed using one or more types of culture media selected from the group consisting of Dulbecco's modified Eagle's medium (DMEM), Iscove's modified Dulbecco's medium (IMDM), alpha modification of Eagle's medium (u-MEM), nutrient mixture F-12 (F12), RPMI 1640, Williams's medium E, McCoy's 5A, and Dulbecco's modified Eagle medium: nutrient mixture F-12 (DMEM/F12), but the present invention is not limited thereto. In addition, the medium may further include at least one of a B27 supplement, an N2 supplement and a G5 supplement. According to one embodiment of the present invention, the culture medium used in co-culture in the present invention may be DMEM, and 10% FBS and/or 1% antibiotics may further be included in the medium.

According to one embodiment of the present invention, the preparation method may further include mixing a normal lung organoid and cancer cells before co-culture and centrifuging the mixture, but the present invention is not limited thereto.

In the lung cancer model of the present invention, the lung cancer may be primary lung cancer or metastatic lung cancer depending on the type of cancer cells fused with the normal lung organoid, but the present invention is not limited thereto. For example, when the cancer cells co-cultured with the normal lung organoid are lung cancer cells, the lung cancer may be a primary lung cancer model, and when the cancer cells co-cultured with the normal lung organoid are not lung cancer cells, the lung cancer may be a metastatic lung cancer model.

In addition, as another aspect, the present invention provides a method of evaluating the efficacy of a lung cancer therapeutic agent, which includes the following steps:
(a) treating a lung cancer model according to the present invention with a lung cancer therapeutic agent; and
(b) when the growth inhibition or apoptosis of the lung cancer model in (a) is increased compared to a control model, determining that the efficacy of the lung cancer therapeutic agent is excellent.

The term “lung cancer therapeutic agent” used in the specification is a material that exhibits an effect of preventing or treating lung cancer, and specifically, refers to a material that can kill lung cancer cells or inhibit the growth thereof.

The term “control model” used in the specification refers to a lung cancer model according to the present invention in a state that is not treated with any material capable of exhibiting an effect of preventing, improving, or treating lung cancer (untreated or non-treated state).

In the specification, the “treatment” may be used interchangeably with “addition” or “administration.”

In the present invention, the lung cancer therapeutic agent may be, but not limited to, a compound, a protein, a fusion protein, a compound-protein complex, a drug-protein complex, an antibody, a compound-antibody complex, a drug-antibody complex, an amino acid, a peptide, a virus, a carbohydrate, a lipid, a nucleic acid, an extract, or a fraction.

The “antibody” used herein includes a monoclonal antibody, a polyclonal antibody, a bispecific antibody, a multispecific antibody, a chimeric antibody, a humanized antibody, and a human antibody, and also includes antibodies already known in the art or commercially available, in addition to novel antibodies. The antibodies include not only a full-length form including two heavy chains and two light chains but also a functional fragment of the antibody molecule. The functional fragment of the antibody molecule refers to a fragment that retains at least an antigen-binding function, and may be Fab, F(ab′), F(ab′)2, or Fv, but the present invention is not limited thereto. The “peptide mimetics” refer peptides or modified peptides biologically mimicking the active ligand of hormones, cytokines, enzyme substrates, viruses or other biological molecules. The “aptamer” means a single-stranded nucleic acid (DNA, RNA or modified nucleic acid) which has a stable tertiary structure and is capable of binding to a target molecule with high affinity and specificity.

In still another example, the lung cancer therapeutic agent may be an antisense nucleic acid, siRNA, miRNA, or ribozyme, which complementarily binds to DNA or mRNA, but the present invention is not limited thereto.

In the present invention, the “antisense nucleic acid” refers to DNA, RNA, or a fragment or derivative thereof, which contains a nucleic acid sequence complementary to the sequence of specific mRNA, and complimentarily binds or is hybridized with the sequence of mRNA to inhibit the translation of mRNA into a protein. The “small interfering RNA (siRNA)” refers to short double-stranded RNA that can induce RNA interference (RNAi) through the cleavage of specific mRNA. siRNA includes a sense RNA strand having a sequence having homology with mRNA of a target gene, and an antisense RNA strand having a sequence complementary thereto. Since siRNA can inhibit the expression of a target gene, it is used in a gene knockdown method or gene therapy method. The “short hairpin RNA (shRNA)” means single-stranded RNA, which is divided into a stem part forming a double-stranded part by hydrogen bonding and a loop part having a ring shape. shRNA may be processed by a protein such as a dicer to be converted into siRNA, and may play the same function as siRNA. The “microRNA (miRNA)” refers to non-coding RNA (21 to 23 nt), which promotes the degradation of target RNA or inhibits the translation thereof, thereby regulating gene expression post-transcriptionally. The “ribozyme” refers to an RNA molecule having the same function as an enzyme that recognizes a specific base sequence and cleaves the sequence. A ribozyme consists of a region that binds to a complementary base sequence of a target messenger RNA strand with specificity and a region that cleaves target RNA.

In the present invention, the growth inhibition or apoptosis in the lung cancer model may be confirmed by an increase/decrease in area of the lung cancer model. In addition, when the area of the lung cancer model decreases, it can be determined that the growth inhibition or apoptosis in an organoid of cancer cells are increased. In addition, the increase/decrease in area of the lung cancer model may be confirmed by a method known in the art, for example, an analytical method such as high-content screening (HCS) or flow cytometry, but the present invention is not limited thereto.

In addition, in still another aspect, the present invention provides a method of screening a candidate material for a lung cancer therapeutic agent, which includes the following steps:
(a) treating the lung cancer model according to the present invention with a candidate material for a lung cancer therapeutic agent; and
(b) when the growth inhibition or apoptosis of the lung cancer model in (a) is increased compared to a control model, determining the candidate material as a lung cancer therapeutic agent.

In the method of screening a candidate material for a lung cancer therapeutic agent according to the present invention, each term is the same as described in the method of evaluating the efficacy of an anticancer agent unless otherwise specified.

The term “candidate material for a lung cancer therapeutic agent” used herein means a material expected to exhibit the effect of preventing or treating lung cancer, and specifically, a material that is expected to kill lung cancer cells or inhibit the growth thereof.

In the present invention, the candidate material may be, but not limited to, a compound, a protein, a fusion protein, a compound-protein complex, a drug-protein complex, an antibody, a compound-antibody complex, a drug-antibody complex, an amino acid, a peptide, a virus, a carbohydrate, a lipid, a nucleic acid, an extract, or a fraction.

Hereinafter, preferred examples are presented to better understand the present invention. However, the following examples are provided to more easily understand the present invention, and the content of the present invention is not limited by the following examples.

### EXAMPLES

### Example 1. Preparation of Lung Organoids

Type 2 alveolar cells were isolated from the lung tissue of each of three different people and used.

The type 2 alveolar cells (1×10⁴ cells) were added to 45 μL of an alveolar medium (alveolar medium, Table 1), suspended and mixed with 45 μL of Matrigel, and then inserted into a 24-Transwell plate for incubation at 37° C. in a CO₂ incubator. After 30 minutes, 10 μM Y27632 was added to 500 μL of alveolar medium on the bottom, after 2 days, the medium was replaced with a fresh alveolar medium every 2 to 3 days, and cultured for 14 to 20 days. The prepared lung organoids were incubated in Matrigel before use.

TABLE 1

| Media component | Final concentration |
|---|---|
| [symbol] CAMP | 100 [symbol] |
| FGF7 (human [symbol] GF) | 10 [symbol] |
| [symbol] | 100 [symbol] |
| B27 supplement | [symbol] |
| [symbol] | [symbol] |
| [symbol] premix | 0.10% |
| BSA | [symbol] |
| CaCl [symbol] | [symbol] |
| H [symbol] | [symbol] |
| Penicillin/Streptomycin | 100 [symbol] /100 [symbol] |
| H [symbol] F12 | 20 [symbol] |

[symbol]

cAMP and PGF7 are added before use. [symbol]
indicates data missing or illegible when filed

### Example 2. Preparation of Lung Cancer Model Using Normal Lung Organoid

To prepare lung cancer models, the lung organoids prepared in Example 1 were isolated from Matrigel. One lung organoid and 100 GFP-tagged or Luc-tagged lung cancer cells (H1299) were put into a tube, and centrifuged at 500 rpm for 1 minute to bring the lung organoid into close contact with the lung cancer cells so that the lung cancer cells adhered to the lung organoid (the number of lung cancer cells may be appropriately adjusted according to the purpose of preparing the model). After centrifugation, the lung organoid and the lung cancer cells in the lower part of the tube were put on a suspension dish containing DMEM supplemented with 10% FBS and 1% antibiotics, and incubated at 37° C. in a CO₂ incubator until the lung cancer cells were fused to the lung organoid.

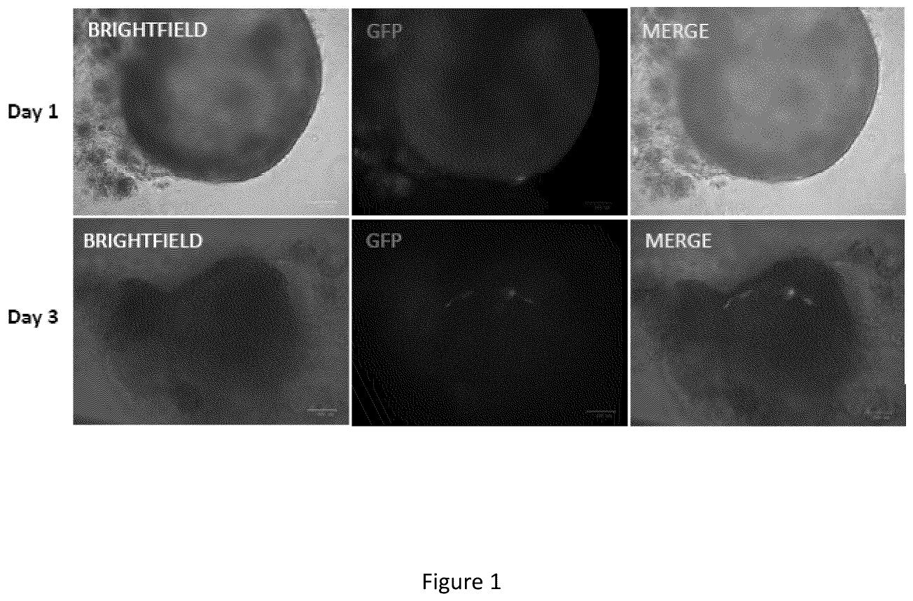

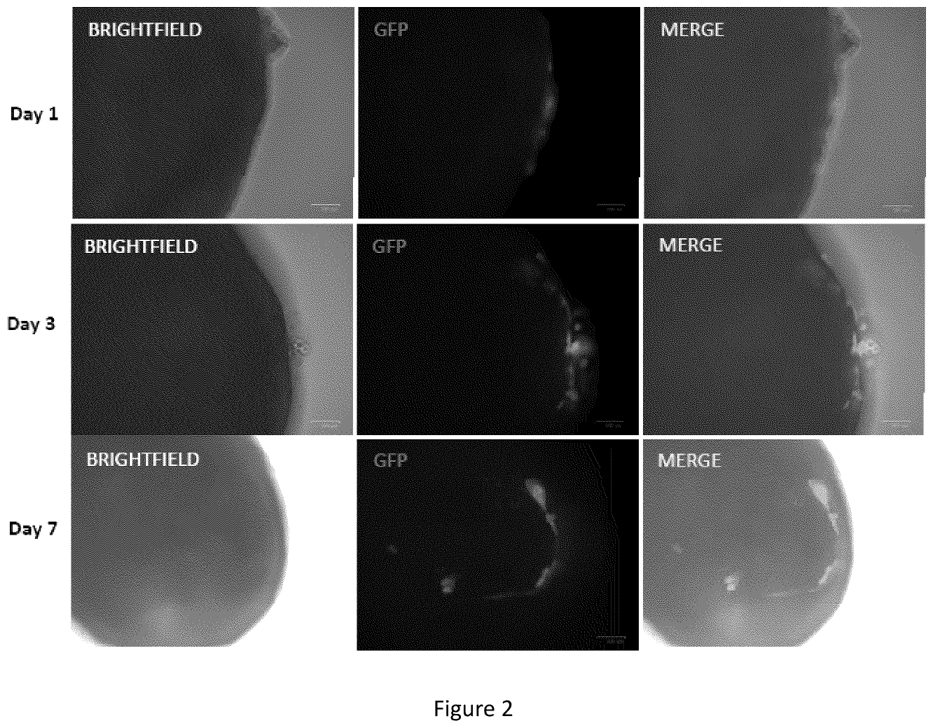

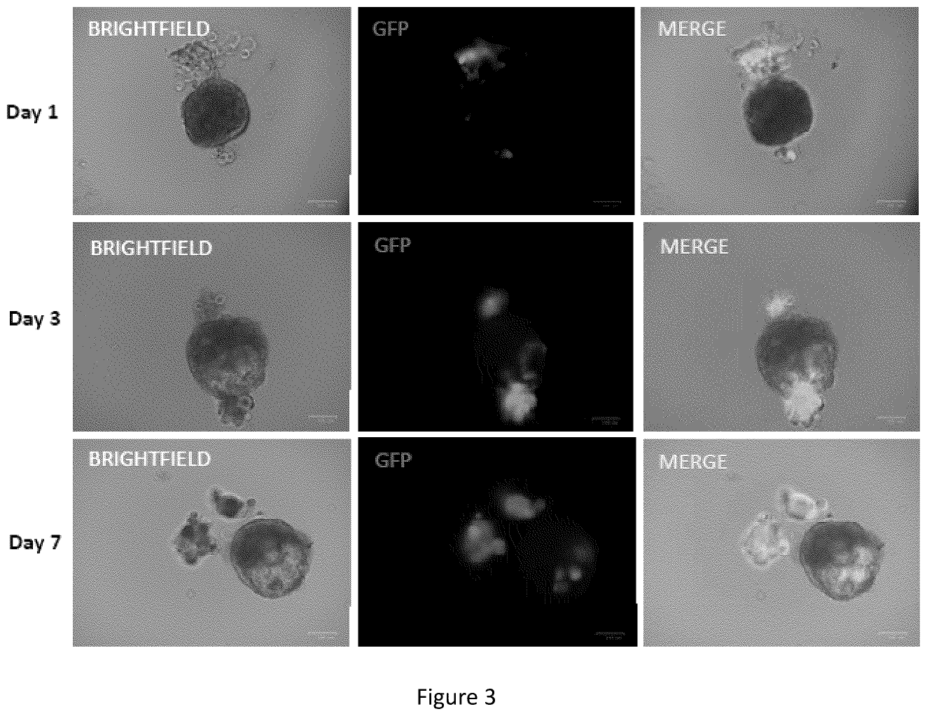

In addition, as shown in FIGS. 1 to 3, on days 1, 3, and 7 of the fusion of a lung organoid derived from the alveolar cells isolated from each of three different people and lung cancer cells, the process of fusing the lung organoid and the lung cancer cells was observed through the GFP of the lung cancer cells.

### Example 3. Identification of Marker of Alveolar Cells in Lung Cancer Model

After the lung cancer model prepared in Example 2 was fixed with 4% paraformaldehyde and subjected to tissue processing, to stain the characteristic factors observed in the lung organoids, the type 1 alveolar cell marker HT1-56, the type 2 alveolar cell marker HT2-280, and the lung fibroblast marker Col1A1 were immunostained.

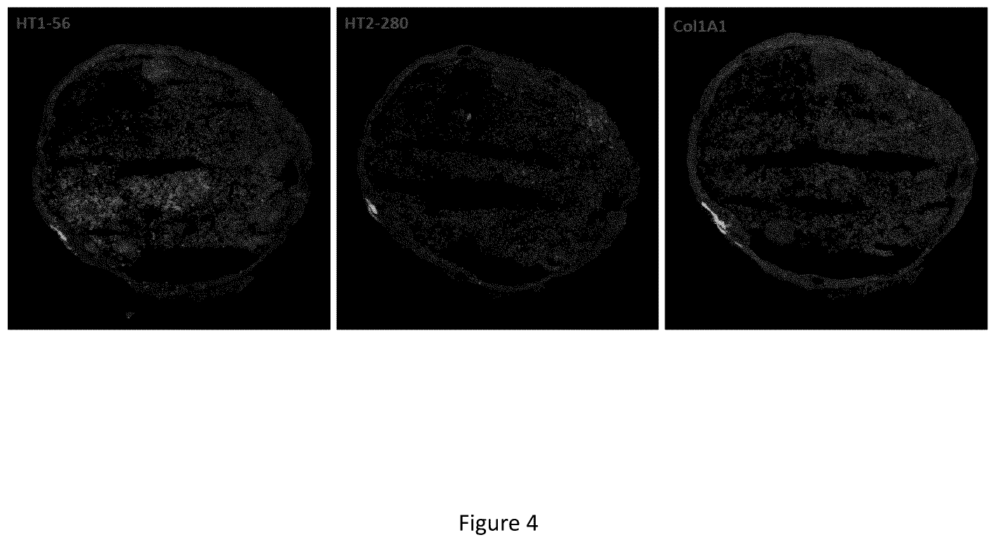

As a result, as shown in FIG. 4, the type 1 alveolar cell marker HT1-56 and the type 2 alveolar cell marker HT2-280, which are components of alveoli, and the lung fibroblast marker Col1A1 were identified.

The present invention relates to a lung cancer model based on a normal lung organoid, prepared by fusing cancer cells to the normal lung organoid. By using the lung cancer model according to the present invention, it is possible not only to observe the progression or metastasis pattern of lung cancer from the early stage of proliferating lung cancer cells to the stage of forming a tumor but also to compare the effect of an anticancer agent at each stage, and thus the information on an appropriate therapeutic agent may be provided according to the stage of cancer development.

In addition, since the lung cancer model according to the present invention includes a normal lung organoid and cancer cells, which are fused, it can be effectively used to confirm the effect of an anticancer agent on normal tissue and the effect of cancer cell-specific apoptosis. In addition, since a conventional method of injecting human lung cancer cells into a mouse has a problem in that unpredictable reactions are caused by immune responses or the like, the present invention is expected to be effectively used as a human lung cancer model.

It should be understood by those of ordinary skill in the art that the above description of the present invention is exemplary, and the exemplary embodiments disclosed herein can be easily modified into other specific forms without departing from the technical spirit or essential features of the present invention. Therefore, the exemplary embodiments described above should be interpreted as illustrative in all aspects and not restrictive.

1. A method of preparing a lung cancer model using a normal lung organoid, comprising:
co-culturing a normal lung organoid and cancer cells.

2. The method of claim 1, wherein the cancer is one or more selected from the group consisting of breast cancer, colorectal cancer, lung cancer, small cell lung cancer, stomach cancer, liver cancer, blood cancer, bone cancer, pancreatic cancer, skin cancer, head and neck cancer, skin melanoma, intraocular melanoma, uterine sarcoma, ovarian cancer, rectal cancer, anal cancer, fallopian tube cancer, endometrial cancer, cervical cancer, vulvar cancer, vaginal cancer, esophageal cancer, small intestine cancer, endocrine cancer, thyroid cancer, parathyroid cancer, adrenal cancer, soft tissue tumors, female urethral cancer, penile cancer, prostate cancer, bronchial cancer, nasopharyngeal cancer, larynx cancer, chronic or acute leukemia, lymphocytic lymphoma, bladder cancer, kidney cancer, ureter cancer, renal cell cancer, renal pelvic cancer, central nervous system (CNS) tumors, primary CNS lymphoma, myeloma, brainstem gliomas, and pituitary adenomas.

3. The method of claim 1, wherein the cancer cells are human primary cancer cells or established cancer cells.

4. The method of claim 3, wherein the established cancer cells are one or more selected from the group consisting of HeLa cells, COLO-201 cells, TE-1 cells, TE-2 cells, TE-3 cells, TE-7 cells, TE-8 cells, TE-12 cells, TE-13 cells, NRC-12 cells, MKN-45 cells, AGS cells, KATO-III cells, Hs746t cells, NCI-N87 cells, HEp-2 cells, Detroit 562 cells, HEC-1B cells, Hs-578T cells, K-562 cells, SF-188 cells, SNU-1 cells, H1299 cells, and WiDr cells.

5. The method of claim 1, wherein the lung organoid and the cancer cells are mixed in a ratio of 1:1 to 1,000 cells.

6. The method of claim 1, further comprising:
mixing the normal lung organoid and the cancer cells before co-culture and centrifuging the mixture.

7. A lung cancer model prepared by the method of claim 1.

8. The model of claim 7, wherein the lung cancer is primary lung cancer or metastatic lung cancer.

9-10. (canceled)

11. A method of screening a candidate material for a lung cancer therapeutic agent, comprising:
(a) treating the lung cancer model of claim 7 with a candidate material for a lung cancer therapeutic agent; and
(b) when the growth inhibition or apoptosis of the lung cancer model in (a) is increased compared to a control model, determining the candidate material as a lung cancer therapeutic agent.

12. The method of claim 11, wherein the growth inhibition or apoptosis of the lung cancer model is confirmed by an increase/decrease in area of the lung cancer model.
